(19) United States
(12) Patent Application Publication
(10) Pub. No.: US 2003/0139582 A1
(43) Pub. Date: Jul. 24, 2003

(54) Composition containing biologically active polypeptides suitable for the oral administration

(76) Inventor: Kai Hua Wang, San Bruno, CA (US)

Correspondence Address: John H. Faro, Esq., Faro & Associates, P.A., P.O. Box 4904, Key Biscayne, FL, 33149-4904, US

(21) Appl. No.: 10/050,017

(22) Filed: Jan. 17, 2002

(51) Int. Cl. A61K 38/18; C07K 14/53

(52) U.S. Cl. 530/399; 514/12

(57) ABSTRACT

A hydrophilic emulsion composition as a carrier fluid for the oral administration of biologically active polypeptides. The composition consists of a commercial therapeutic polypeptide product, e.g., Granulocyte Colony Stimulating Factor (G-CSF), and a carrier fluid containing a small molecule spleen extract and a fluid mixture of substances that are complimentary to said small molecule extract to protect the polypeptide and promote the absorption of the polypeptide by epithelium of intestinal mucosa.

### BACKGROUND OF THE INVENTION

1. Field of the Invention

This invention relates to a composition of matter, the process for the preparation thereof and to use of such composition in the delivery of therapeutic substances. More specifically, this invention is directed to a carrier fluid formulation suitable for oral administration of a biologically active polypeptide, to a method for the incorporation of a biologically active polypeptide into a convenient and effective liquid carrier medium and to a method for the oral administration of a biologically active polypeptide. The carrier fluid formulation comprises substances that protect the polypeptide and promote the absorption of the polypeptide by epithelium of intestinal mucosa.

2. Description of the Prior Art

The therapeutic use of biologically active polypeptides has traditionally required their administration intravenously because of the economics and efficacy concerns associated with their use. More specifically, biologically active polypeptides are generally vulnerable to metabolism in the gut if administered orally, or can trigger an immune response. Thus, the amount of such biologically active polypeptides that are prescribed for oral administration must necessarily be increased to overcome such natural processes. Accordingly, the oral administration is generally inherently less effective and/or prohibitively expensive since the dosage must be increased to compensate for such natural processes.

Human granulocyte colony stimulating factor (also hG-CSF or G-CSF) is one of these biologically active polypeptide. This polypeptide is known to exhibit haematopoietic growth factors. It has also been shown to be present in the conditioned medium of a human bladder carcinoma cell line denominated 5637 (ATCC HT8-9) (Welte et al., Proc. Natl. Acad. Sci. (USA), 82, pp.1526-1530, (1985)). Moreover, the determination of a DNA sequence encoding human G-CSF (Japanese Patent Application Laying Open KOHYO No. 500636/88) has enabled the production of human G-CSF by means of recombinant genetic techniques.

It has been reported that human G-CSF may be useful in (a) treatment of general haematopoietic disorders including those arising from chemotherapy or from radiation therapy; (b) bone marrow transplantation therapy; (c) wound healing in burn treatment therapy; and, (d) the treatment of bacterial inflammation, Welte et al., supra.

Unfortunately, such physiologically-active proteins, such as human G-CSF, when administered orally, retain their pharmacological activity for only for a short period of time due to their high clearance rate in body. In addition, the high hydrophobicity of the human G-CSF reduces its stability. Because of the therapeutic potential of human G-CSF, a number of efforts have been made to overcome one or more of the polypeptide's shortcomings. More specifically, chemical modification of human G-CSF by modification with polyethylene glycol reportedly improves its in vivo stability and reduces its immunogenicity.

Compared with conventional injection methods, oral administration of therapeutic polypeptides has a number of significant advantages. For example, such oral formulations are more convenient to use, and do not present the common side effects of injection, e.g., muscle or bone pain, and fever, etc., and is cost effective. Accordingly, orally administered therapeutic polypeptides have been the focus of research and development by the pharmaceutical companies and institutions, and numerous reports and patents have been published on related topics involving their preparation and use.

Notwithstanding such efforts, additional work is required to develop and refine the delivery of the such orally administered therapeutic polypeptides, including specifically, human G-CSF, to make such orally administered therapeutics more competitive with dosage levels normally administered in injection delivery systems.

### OBJECT OF THE INVENTION

It is the object of this invention to remedy the above as well as related deficiencies in the prior art.

More specifically, its is the principle object of this invention to provide a therapeutic composition having a biologically active polypeptide and a fluid carrier that can be administered orally.

It is another object of this invention to provide a therapeutic composition having a biologically active polypeptide and a fluid carrier that can be administered orally at a dosage level that is competitive with administration by injection.

It is still yet another object of this invention to provide a therapeutic composition having a biologically active polypeptide and a fluid carrier that can be administered orally at a dosage level that is relatively stable and resistance to clearance prior to absorption.

Additional objects of this invention include a method for the preparation of a formulation containing a biologically active polypeptide suitable for oral administration, and a method for administration of such formulation

### SUMMARY OF THE INVENTION

The above and related objects are achieved by providing a therapeutic composition comprising a biologically active polypeptide (e.g., Human granulocyte colony stimulating factor, also “hG-CSF” or “G-CSF”) and fluid carrier suitable for oral administration.

The fluid carrier of the composition of this invention comprises a mixture having

(a) an emulsifier specific for the biologically active polypeptide,

(b) a immunogenicity suppression agent to reduce the body's humoral and/or cellular response to the biologically active polypeptide, so as to prevent inactivation of the biologically active polypeptide, and

(c) an emulsion stabilizer to preserve the suspension of the biologically active polypeptide within the fluid carrier

In the preferred embodiments of this invention, the oral composition of this invention is readily absorbable by the epithelium of the intestinal mucosa, and once absorbed remains within the intercellular and intracellular integument for an extended period. While the precise mechanism by which the individual ingredients of the composition cooperate is not known, it is hypothesized that stabilization of the biologically active polypeptide within the carrier both preserves the polypeptide activity upon its absorption from the gut into the systemic system; and, otherwise protects the biologically active polypeptide from the body's natural process until delivery to the target tissue. Upon delivery to the target tissue, the biologically active polypeptide is transported across the plasma membranes of the cells and organelles whereby it provides its intended therapeutic benefit. It is this combination of effects which enables oral administration of the biologically active polypeptide at a dosage that is effective at a level that is both economic and competitive to other modalities of administration (e.g. injection).

In one of the preferred embodiments of this invention, the biologically active polypeptide, G-CSF, is conjugated with Vitamin B12 to enhance its biologically activity within the formulation of this invention. While the precise mechanism is not known with certainty, it is hypothesized that the conjugation G-CSF to Vitamin B12 enhances the bioavailability of the biologically active peptide and, thus, accounts for the demonstrative increase in neutrophil population.

In another of the preferred embodiments of this invention, the biologically active polypeptide, G-CSF, is combined with a carrier containing minute amounts of ethylene diamine tetra-acetate (EDTA). The resulting formulation has enhanced resistance to metabolism/enzymatic conversion of the polypeptide, thus, increasing the amount of available polypeptide for absorption into the target tissues. The addition of EDTA to the formulation is believed to inhibit the enzymes by chelating with the metal co-factors needed to effect such enzymatic conversions.

### DETAILED DESCRIPTION OF THE INVENTION INCLUDING PREFERRED EMBODIMENTS

The present invention relates to a pharmaceutical composition designed for oral administration. This composition contains a biologically active polypeptide having therapeutic properties, and a fluid carrier that facilitates the delivery of the polypeptide and promotes its absorption by the body of the patient.

The carrier fluid is strategically comprised of a small molecule extract from animal spleen organs (SE) along with other complementary substances, such as nonfat milk (M), polyvinylpyrrolidone (PVP) and lecithin (L), the details thereof being set forth in the Examples. In one of the preferred embodiments of this invention, the carrier fluid also contains ethylene diamine tetra-acetate (EDTA). In another of the preferred embodiments of this invention, the biologically active polypeptide is conjugated to Vitamin B12.

MLE Component Of Carrier Fluid—Non-fat Milk, PVP, and Lecithin are safe and commonly used additives in food industries. They are also commonly used in the fields of biochemistry and organic chemistry as emulsifying, protecting, or stabilizing agent. Similarly, EDTA is also known as chelating agent that can bind to ions (metal ions) and thereby protect the biologically active polypeptides from various enzymes and other endogenous agents which can result in its clearance and/or inactivation. The component of the carrier fluid containing the foregoing is also herein referred to as “MLE”.

This MLE component of the carrier fluid can be prepared initially by forming a suspension/emulsion containing 1% milk powder in distilled water, and the mixture stirred at room temperature until dissolved. The stirring is continued for about 4 hours or until a uniform and stable emulsion is formed. At this juncture, lecithin powder is added and stirring continued until the lecithin is thoroughly dissolved. The relative concentration of lecithin in the carrier fluid is in the range of from about 0.25 to about 1.0 weight percent. PVP can also be added to the emulsion in a like amount. In the preferred embodiments of this invention, the carrier also contains about 1 mM EDTA. The resultant carrier fluid is stirred until a semi-transparent final product is obtained. The carrier fluid can be stored at −20° C., or lyophilized and stored at 4° C. and reconstituted as needed

SE Component of Carrier Fluid—The extract from animal or cattle spleen organs (SE) is obtained by means of dialysis or ultra filtration using a cutoff of MW14000. The product of extraction mainly consists of peptides, nucleotides, and other substances with a molecular weight less than 14000. The SE solution is measured at UV 260 nm using spectrophotometer. OD value of 1.0 represents approximately 50 ug/ml.

Listed below are some of the functions of carrier fluid which promote the delivery of biologically active polypeptides in the composition of this invention, including specifically, the absorption thereof into the circulation system via the epithelium of intestinal mucosa:

Emulsification of the biologically active polypeptide so that it becomes easier to be absorbed by the small intestine;

Blockage and reduction of the body's clearance mechanism against the biologically active polypeptide; and

Stabilization of the polypeptide by maintaining it in colloidal suspension within the carrier.

It is also believed that lecithin component of the carrier fluid assists in the transport of the polypeptide across the plasma membranes of the cells and organelles of the body that can be benefited by such polypeptide (e.g., target tissues).

### EXAMPLES

The Examples which follow further describe, define and illustrate a number of the preferred embodiments of this invention. Parts and percentages appearing in such Examples are by weight unless otherwise indicated. Apparatus and equipment used in the formulation, analysis and evaluation of the inventions are standard unless indicated otherwise.

### Example I

As noted herein in the Background of the Invention, human G-CSF is one of the haematopoietic growth factors. The following experiment demonstrates the effect of the oral delivery system on Granulocyte Colony Stimulating Factor (G-CSF) in generating neutrophiles on cyclophosphamide (CTX)-induced neutropenic C57/BL mice (Table 1).

TABLE 1
Pharmacological activity of hG-CSF on cyclophosphamide
(CTX)-induced neutropenic C57/BL mice, administered both
subcutaneously (S.C.) and orally (Oral) with SMPL formula.

| | Number of Neutrophiles/mm³ | | |
|---|---|---|---|
| | G-CSF | G-CSF | 4 days after |
| Groups | after 8 hrs. | after 96 hrs. | stopping G-CSF |
| CTX 0.16 mg/g/B.W./day | 533 | 483 | 8000 |
| CTX + G-CSF 4 ug/ea S.C. | 1167 | 6250 | 6300 |
| CTX + G-CSF 36 ug/ea | 2033 | 11000 | 12733 |
| Oral | | | |

As is evident for the observed data reported in Table 1, oral or subcutaneous administration of G-CSF to CTX induced neutropenic C57/BL mice, at dosage of 0.16 mg/g/B.W, shows an increased level of neutrophiles after 8 hours. Increases of neutrophiles were also observed after 96 hours of administering G-CSF. After 4 days, however, the effect of the orally administered G-CSF lasted longer in the host's body than the comparable subcutaneously administered polypeptide; and, the level of neutrophiles of the orally administered G-CSF remained high, while neutrophiles level of administered subcutaneously group didn't even reach level of CTX group. The results suggest that the oral G-CSF composition will be more stable in the body during therapeutic process, and therefore, G-CSF can be given to the patient less frequently than what would be necessary for injection methods.

The same results can be observed in the experiments using C57/BL, Balb/c and Swiss Webster mice. Furthermore, the long lasting stability of the oral composition is shown 2 days, 3 days, and 4 days is apparent even after the administration of the G-CSF has been discontinued.

Comparative studies demonstrate the effectiveness of the oral composition in increasing the level of neutrophiles in normal Swiss Webster mice (male, 7 week of age) after 8 and 26 hours respectively. These comparative studies are summarized in Table 2, which appears below.

TABLE 2
Pharmacological activity of hG-CSF on normal Swiss Webster mice,
administered subcutaneously (S.C.) and orally (Oral) with SMPL formula.

| | Number of Neutrophiles/mm³ | |
|---|---|---|
| Groups | G-CSF after 8 hrs. | G-CSF after 26 hrs. |
| Normal Control | 7285 | 17000 |
| G-CSF 0.2 ug/g/B.W. (SC) | 11806 | 19994 |
| G-CSF 2.0 ug/g/B.W. (Oral) | 9817 | 38177 |

As is evident for the observed data reported in Table 2, the oral administration of G-CSF increases level of neutrophiles by 20%, even with control base level of neutrophiles as high as 7285 per mm³. Such 20% increase is evident after 8 hours oral administration, and a 124% increase after 26 hours.

### Example 2

The following comparative study demonstrated the functions of SMPL oral delivery formula.

TABLE 3
Pharmacological activity of the delivery formula for G-CSF
on cyclophosphamide CTX-induced neutropenic Balb/c mice

| | Number of Neutrophiles/mm³ | |
|---|---|---|
| Groups | G-CSF after 72 hrs. | G-CSF after 120 hrs. |
| CTX | 583 | 15950 |
| CTX + G-CSF in PB | 666 | 15368 |
| CTX + G-CSF in SMPL | 1383 | 23900 |

TABLE 4
Pharmacological activity of the delivery solution for G-CSF
on cyclophosphamide (CTX)-induced neutropenic C57/BL mice

| | Number of Neutrophiles/mm³ | |
|---|---|---|
| | G-CSF | G-CSF |
| Groups | after 48 hrs. | after 120 hrs. |
| CTX | 533 | 2400 |
| G-CSF S.C. 0.16 ug/g/B.W. | 1167 | 3125 |
| G-CSF Oral 1.28 ug/g/B.W. in SMPL | 2033 | 5500 |
| G-CSF Oral 1.28 ug/g/B.W. in SCPL | 1850 | 4375 |
| G-CSF Oral 1.28 ug/g/B.W. in SB | 1483 | 3450 |

As is evident for the observed data reported in Table 2, the number of neutrophiles of CTX neutropenic mice is similar to those using G-CSF in phosphate buffer after 72 hours. However, the level of neutrophiles of G-CSF-SMPL group is more than 2.3 times higher than the CTX group. The level of neutrophiles of G-CSF-SB group is also significantly higher than that of CTX group, but it was the SMPL group that scored the highest level, which is more than 3.8 times over CTX group. SCPL is SMPL substituting milk with casein; it did not quite match the level of SMPL. Accordingly, the G-CSF SMPL is the preferred carrier fluid composition.

### Example 3

The following dosage comparison study was designed in order to indicate the best dosage of G-CSF oral administration. Using cyclophosphamide (CTX)-induced neutropenic Balb/c mice as hosts, 0.16 ug/g/B.W., CTX injection was made daily for 3 days. Beginning on day 4, the hosts were administered with G-CSF with different dosages for 4 days. Observations are recorded at 48 hours, 96 hours, and 120 hours after administering G-CSF, and 4 day after stopping the administration of G-CSF.

TABLE 5
Pharmacological activity of G-CSF on cyclophosphamide
(CTX)-induced neutropenic Balb/c mice compared by
different dosages and types of administrations.

| | Number of Neutrophiles/mm³ | | | |
|---|---|---|---|---|
| Groups | 48 hrs. | 96 hrs. | 120 hrs. | Stop 4 days |
| CTX | 1018 | 3225 | 5700 | 9267 |
| CTX + G-CSF S.C. | 1381 | 14100 | 24306 | 10038 |
| 0.12 ug/g/B.W./day | | | | |
| CTX + G-CSF S.C. | 1650 | 31716 | 51762 | 15916 |
| 0.24 ug/g/B.W./day | | | | |
| CTX + G-CSF Oral | 730 | 4925 | 6375 | 13800 |
| 0.8 ug/g/B.W./day | | | | |
| CTX + G-CSF Oral | 1550 | 13663 | 20183 | 13771 |
| 1.6 ug/g/B.W./day | | | | |

As is evident for the observed data reported in Table 5,

(a) When the oral dosage was 4 times larger that injection dosage, there wasn't any obvious increase of neutrophil level; however, when the oral dosage was 8 times larger than the injection dosage, the oral group had the similar neutrophil level as the injection group.

(b) The lower oral dosage corresponds to a slower increase of neutrophiles; after the administration was stopped, however, the number of neutrophil remained at the similar level as the higher oral dosage.

(c) Most significantly, after the administration stopped for days, there was a steady increase of neutrophil level with the oral groups, while there was a significant drop with the injection groups.

The above data suggests that SMPL is an ideal oral delivery formula for G-CSF and other pharmacological polypeptides.

### Example 4

G-CSF like other biologically functional polypeptides is very unstable and has high clearance rate in the body. It has been reported that the half-life of G-CSF in the body of normal person or cancer patient is about 3.5 hours; its clearance rate being in the range of from about 0.5-0.7 mg/min./kg.

It was observed that male ICR mice, subcutaneously administered with G-CSF at the dosage of 10 ul/kg, reached the highest level of neutrophiles in 6 to 12 hours after the injection. It would gradually decrease after 12 hours, and finally reach the basal level after 30 hours. The oral G-CSF composition with SMPL formula, however, can remain stable in the host's body. The level of neutrophiles changed steadily and remained at higher than basal level even 3 days after administration was stopped.

TABLE 6
Comparative Study on the Stability of Pharmacological activity of
G-CSF on cyclophosphamide (CTX)-induced neutropenic Balb/c mice.

| | Number of Neutrophiles/mm³ | | | |
|---|---|---|---|---|
| | | | | 3 days |
| Groups | 24 hrs. | 48 hrs. | 96 hrs. | after stop |
| CTX 0.16 mg/g 3 days | 863 | 683 | 2563 | 11563 |
| CTX + G-CSF S.C. | 1020 | 700 | 9850 | 11700 |
| 0.2 ug/g/B.W. | | | | |
| CTX + G-CSF Oral | 1071 | 905 | 5642 | 15029 |
| 1.6 ug/g/B.W. | | | | |

### Example 5

The carrier fluid is now formulated with ethylene diamine tetra-acetate (EDTA) as a protecting agent for G-CSF in the oral composition. As previously noted herein, EDTA is a chelating agent that binds with ions, which will form protection for a polypeptide like G-CSF against the clearance by the enzymes present in the host's body. Only a trace amount (1 mM/mg) of EDTA is necessary to be added to G-CSF for protection purposes. EDTA is considered safe, as its typical oral dosage for the treatment of lead poisoning can be as high as 3 gram/day. (Proc 13 Int Congr. Health, N.Y., 1960 (1961) LT. Petrovic MD. Etc.)

The following experiment was designed to verify EDTA's function of protecting G-CSF. Using cyclophosphamide (CTX)-induced neutropenic Balb/c mice as hosts, 0.16 ug/g/B.W. CTX injection was made twice in a row. G-CSF and G-CSF with EDTA were orally administered respectively. Then CTX was injected for the third time. Thus the oral G-CSF composition was administered for four time continuously. Observations were made at 48 and 96 hours after G-CSF was orally administered.

TABLE 7
Pharmacological activity of G-CSF and G-CSF-B12 on
cyclophosphamide (CTX)-induced neutropenic Balb/c mice.

| | Number of Neutrophiles/mm³ | |
|---|---|---|
| | G-CSF | G-CSF |
| Groups | after 48 hrs. | after 96 hrs. |
| CTX | 583 | 15950 |
| G-CSF-EDTA in SMPL 1.4 ug/g/B.W. | 1475 | 26650 |
| G-CSF in SMPL 1.4 ug/g/B.W. | 935 | 23900 |

As is evident from the data reported in Table 7, adding EDTA to G-CSF significantly increased the level of neutrophiles compared with regular G-CSF group.

### EXAMPLE 6

Vitamin B12 is now conjugated to the biologically active polypeptide to determine the effect thereof on the pharmacological activity of G-CSF. Accordingly, 1-ethyl-3-(3-dimethylaminopropyl) carbodimide was used to form covalent bond between Vit. B 12 and G-CSF prior to mixing with SMPL oral formula. Normal Swiss Webster mice with high basal level of neutrophil were used to demonstrate the increase of neutrophiles by the oral administration of G-CSF-Vit. B12 as compared with the results of regular G-CSF administered orally and subcutaneously.

TABLE 8
Pharmacological activity of the delivery
solution for G-CSF on normal Swiss Webster mice.

| | Number of | |
|---|---|---|
| | Neutrophiles/mm³ | |
| | G-CSF | G-CSF |
| Groups | after 16 hrs. | after 24 hrs. |
| Control | 8500 | 9783 |
| G-CSF S.C. 0.2 ug/g/B.W. | 15383 | 13733 |
| G-CSF with SMPL Oral 1.6 ug/g/B.W. | 10383 | 11317 |
| G-CSF-B12 with SMPL Oral 1.6 ug/g/B.W. | 17183 | 14100 |

As is evident from the data reported in Table 8, the oral G-CSF-Vit. B12 group had 100% increase of neutrophiles in 16 hours. The result is significant because of the higher basal level.

The manufacture of recombinant G-CSF (Amgen) has recommends a normal injection dosages of 5 ug/kg, 10 ug/kg, or 20 ug/kg, depending upon individual patient's needs. No drug accumulation effect has been found from continuous administration of G-CSF. Dosage as high as 138 ug/kg/day did not cause toxicity related side effect. Our experiment on mice also showed there was none toxicity effect at dosage of 3.45 ug/g/B.W.

Typical dosages of oral administration are very high. Published recommended dosage of oral G-CSF administration that is up to 1000 times higher than that of injection has been reported (U.S. Pat. No. 6,166,183, Ishikawa et al). The dosage of the present oral composition, on the other hand, is no more than 8 times higher than injection dosage, and its superior stability has clear advantages over injection because it has a much longer lasting neutrophil-increasing activity than that of the injected G-CSF, enabling fewer numbers of administration with a lower dose than normal oral administration.

What is claimed is:

1. In a therapeutic composition containing a biologically active polypeptide and a carrier fluid, which is suitable for oral administration, wherein the improvement comprising:
a homogenous carrier fluid containing
A. A small molecule extract derived from an animal spleen obtained by dialysis or ultra filtration of a tissue homogenate of said spleen, said small molecules of said spleen extract having an average molecular weight of about 14000, or less, and containing peptides, nucleotides, and other tissue derived substances, said extract being further characterized as having a peak absorption at 260 nm, and an optical density of 1.0, which is indicative of approximately 50 ug of extract per milliliter.
B. A fluid mixture of substances that are complimentary to said small molecule extract comprising,
(1) an emulsifier specific for the biologically active polypeptide,
(2) a immunogenicity suppression agent to reduce the body's humoral and/or cellular response to the biologically active polypeptide, so as to prevent inactivation of the biologically active polypeptide, and
(3) an emulsion stabilizer to preserve the suspension of the biologically active polypeptide within the fluid carrier

2. The improved therapeutic composition of claim 1, wherein the biologically active polypeptide is Granulocyte Colony Stimulating Factor (G-CSF).

3. The improved therapeutic composition of claim 1, wherein the biologically active polypeptide is a Granulocyte Colony Stimulating Factor (G-CSF) analog.

4. The improved therapeutic composition of claim 1, wherein the biologically active polypeptide is a Granulocyte Colony Stimulating Factor (G-CSF) and said polypeptide is conjugated to Vitamin B12.

5. The improved therapeutic composition of claim 1, wherein the carrier fluid contains ethylene diamine tetra-acetate (EDTA).

6. In a method for administration of a therapeutic composition containing a biologically active polypeptide and a carrier fluid, wherein the improvement comprising:
A. suspension of said biologically active polypeptide in a homogenous carrier fluid containing
(1). A small molecule extract derived from an animal spleen obtained by dialysis or ultra filtration of a tissue homogenate of said spleen, said small molecules of said spleen extract having an average molecular weight of about 14000, or less, and containing peptides, nucleotides, and other tissue derived substances, said extract being further characterized as having a peak absorption at 260 nm, and an optical density of 1.0, which is indicative of approximately 50 ug of extract per milliliter.
(2). A fluid mixture of substances that are complimentary to said small molecule extract comprising,
(a) an emulsifier specific for the biologically active polypeptide,
(b) a immunogenicity suppression agent to reduce the body's humoral and/or cellular response to the biologically active polypeptide, so as to prevent inactivation of the biologically active polypeptide, and
(c) an emulsion stabilizer to preserve the suspension of the biologically active polypeptide within the fluid carrier
B. Orally administering a therapeutic effective amount of said suspension of Step (A).

7. The improved method for administration of a therapeutic composition of claim 6, wherein the biologically active polypeptide is Granulocyte Colony Stimulating Factor (G-CSF).

8. The improved method for administration of a therapeutic composition of claim 6, wherein the biologically active polypeptide is a Granulocyte Colony Stimulating Factor (G-CSF) analog.

9. The improved method for administration of a therapeutic composition of claim 6, wherein the biologically active polypeptide is a Granulocyte Colony Stimulating Factor (G-CSF) and said polypeptide is conjugated to Vitamin B12.

10. The improved method for administration of a therapeutic composition of claim 6, wherein the carrier fluid contains ethylene diamine tetra-acetate (EDTA).
